(19) United States
(12) Patent Application Publication
(10) Pub. No.: US 2015/0056297 A1
(43) Pub. Date: Feb. 26, 2015

(54) NUTRACEUTICAL COMBINATION FOR PREVENTION AND TREATMENT OF TYPE 2 DIABETES

(71) Applicant: Charles H. Liu, Novi, MI (US)

(72) Inventor: Charles H. Liu, Novi, MI (US)

(21) Appl. No.: 14/461,533

(22) Filed: Aug. 18, 2014

Related U.S. Application Data
(60) Provisional application No. 61/959,495, filed on Aug. 26, 2013.

(51) Int. Cl. A61K 36/82; A61K 33/06; A61K 33/24; A61K 31/155; A61K 36/258; A61K 36/54; A61K 31/047; A61K 38/28; A61K 33/30; A61K 31/714

(52) U.S. Cl. CPC A61K 36/82; A61K 33/30; A61K 33/06; A61K 33/24; A61K 31/714; A61K 36/258; A61K 36/54; A61K 31/047; A61K 38/28; A61K 31/155
USPC 424/643

(57) ABSTRACT

A dietary supplement in capsule or tablet form includes a source of zinc in an amount equivalent to 6 mg to 40 mg of zinc chloride; optionally a source of chromium in an amount equivalent to 100 micrograms to 1000 micrograms of chromium picolinate; optionally from 4 micrograms to 100 micrograms of Vitamin B12; optionally an extract or powdered form of American Ginseng in an amount that provides from 20 mg to 200 mg of ginsenoside(s), or 20 mg to 200 mg of isolated or synthesized ginsenoside(s); optionally an oil, extract or powdered form of cinnamon bark in an amount that provides 1000 mg to 5000 mg of methylhydroxychalcone polymer; optionally 2 mg to 50 mg lutein; and optionally green tea extract in an amount that provides from 200 mg to 4000 mg of epigallocatechin gallate or 200 mg to 4000 mg of isolated or synthesized epigallocatechin gallate.

### CROSS-REFERENCE TO RELATED APPLICATIONS

This application claims the benefit of U.S. Provisional Application No. 61/959,495, filed Aug. 26, 2013, which is incorporated herein by reference.

### FIELD OF THE DISCLOSURE

This disclosure relates to a dietary supplement for controlling the glucose level in the blood of humans and other mammals, and a method for prevention, ameliorating or treating Type 2 diabetes.

### BACKGROUND OF THE DISCLOSURE

Diabetes is a disease in which the body does not produce or properly use insulin. Insulin converts sugar into energy so it is critical to the health of every organ. Diabetes can lead to a wide range of other serious health complications, including heart disease, high blood pressure, blindness and kidney disease. Type 2 diabetes accounts for 90-95% of all cases of diabetes, and is generally associated with obesity, family history, lack of exercise and age. Type 1 diabetes, representing 5-10% of all cases, is genetic in origin and most patients in this category are administered insulin. The disclosed supplement is not directed to this audience. In the United States, there are 21 million people diagnosed with Type 2 diabetes and at least 7 million that are undiagnosed. It is the fastest growing disease in the U.S. and is the fifth leading cause of death. It is estimated that 33% of Caucasian Americans will develop diabetes. Asian-Pacific and African-Americans have higher risks.

The costs of treating diabetes globally was estimated to be $548 billion in 2013, and to be $245 billion in the United States in 2012. Diabetes treatment represents about 11% of the U.S. healthcare expenditure and is only expected to grow as more people move into the high-risk segments. While healthcare experts believe that most Type 2 diabetes is preventable and reversible, diets with large portions and processed preparations combined with sedentary lifestyles make prevention and reversal difficult.

Only 10% of the current diabetes drugs are considered as frontline in reducing glycated hemoglobin (Hb A1c) levels by 1% or more. Every 1% reduction in Hb A1c, an indicator of blood glucose levels, reduces the impact of death due to diabetes by 20%. The limitations of most prescription drugs are that they are expensive ($150 or more per month), the medication efficacy decreases over time, side-effects are problematic (several class action lawsuits), and many medications do not support the energy required to exercise—50% of the drugs are weight neutral at best and about 50% will cause weight gain. Obesity hinders the effectiveness of diabetes treatments.

### SUMMARY OF THE DISCLOSURE

The dietary supplements for treating, preventing or ameliorating Type 2 diabetes can include, in a twice daily dosage form, an orally administrable source of magnesium in an amount that is the equivalent of from 10 mg to 400 mg of magnesium chloride; an orally administrable source of zinc in an amount that is the equivalent of from 3 mg to 20 mg of zinc chloride; optionally an orally administrable source of chromium in an amount that is the equivalent of up to 500 micrograms of chromium picolinate; optionally Vitamin B12 in an amount up to 100 micrograms; optionally an extract or powdered form of American Ginseng in an amount that provides up to 100 mg of ginsenosides; optionally an oil, extract or powdered form of cinnamon bark in an amount that provides up to 2500 mg of methylhydroxychalcone polymer; optionally lutein in an amount up to 25 mg; and optionally green tea extract in an amount that provides up to 2000 mg of epigallocatechin gallate.

In certain aspects of this disclosure, isolated or synthesized ginsenoside(s) can be substituted for the natural extracts or powdered forms of American ginseng.

In certain aspects of this disclosure, isolated or synthesized methylhydroxy chalcone polymer can be substituted for the oil, extract or powdered form of cinnamon bark.

In certain aspects of this disclosure, isolated or synthesized epigallocatechin gallate can be substituted for green tea extract.

The methods for treating, preventing or ameliorating Type 2 diabetes can include orally administering to a patient on a daily basis via one or a plurality of tablets or capsules at a single time or multiple times throughout the day, a source of magnesium in an amount equivalent to 20 mg to 800 mg of magnesium chloride; a source of zinc in an amount equivalent to 6 mg to 40 mg of zinc chloride; optionally a source of chromium in an amount equivalent to 100 micrograms to 1000 micrograms of chromium picolinate; optionally from 4 micrograms to 200 micrograms of Vitamin B12; optionally an extract or powdered form of American ginseng in an amount that provides from 20 mg to 200 mg of ginsenoside(s), or 20 mg to 200 mg of isolated or synthesized ginsenoside(s); optionally an oil, extract or powdered form of cinnamon bark in an amount that provides 1000 mg to 5000 mg of methylhydroxychalcone polymer, or 1000 mg to 5000 mg of isolated or synthesized methylhydroxychalcone polymer; optionally 2 mg to 50 mg lutein; and optionally green tea extract in an amount that provides from 200 mg to 4000 mg of epigallocatechin gallate or 200 mg to 4000 mg of isolated or synthesized epigallocatechin gallate.

These and other features, advantages and objects of the various embodiments will be better understood with reference to the following specification and claims.

### DETAILED DESCRIPTION

Men and women at an age of about 50 or greater that have been diagnosed with pre-diabetes or full diabetes are likely to benefit from administration of the disclosed dietary supplements. Such individuals are advised to change their diet and to increase exercise. Generally, only individuals diagnosed with full diabetes are given a drug prescription, often Metformin. However, recent studies have confirmed that metformin is not an effective drug for men who are at risk of heart diseases, a common condition among diabetic patients. As a result, alternative or lower metformin dose treatments can be particularly helpful for men.

The disclosed dietary supplement is safe to use for pre-diabetes patients to delay or stop the progression of symptoms. The supplement can be prescribed, recommended, or self-administered as an adjunct therapy or to promote better health through appropriate regulation of glucose metabolism. Based on the results of our phase I clinical testing, we estimate that the disclosed dietary supplement can reduce Hb A1c in the range of 0.10-1.00 when added to the existing therapy regimen and will provide patients with other favorable and measurable results such as weight loss, blood pressure reduction, and increased energy.

The disclosed dietary supplement formulation for Type 2 diabetes can be administered to patients to increase energy, improve vision, boost immune system, promote sugar metabolism, and increase lean body mass. The formulation can comprise Chromium Picolinate, Magnesium Chloride, Vitamin 1312, Zinc Chloride, and an extract comprising American Ginseng, cinnamon bark, Lutein and Green tea. An example of a single dose tablet or capsule, which may be taken twice daily, can comprise 50-500 mcg of Chromium Picolinate, 10-100 mg of Magnesium Chloride, 2-10 mcg Vitamin B12, 3-20 mg Zinc Chloride, and 250 mg-2000 mg of extract comprising American Ginseng, Cinnamon Bark, Lutein and Green tea. Such compositions containing all of these components in amounts specified by the ranges have been designated, and are expected to be marketed under the name, “DiabetainC”. The formulation comprises ingredients that exhibit stability over time when combined with metformin in one tablet or capsule. The combination of herbs with metformin or any other prescription medications for treating diabetes in a tablet or capsule to maximize the prescription effects or minimize the side effects is believed to be novel. The formulation can be used as an adjunct therapy to delay diabetes progression and help control the blood glucose. It can be used as an adjunct therapy to metformin to help control the blood sugar and delay the addition of a second adjunct prescription medication. The formulation can be administered to individuals over 50 years old who are at risk with heart diseases, and may be especially beneficial for treating men to reduce metformin dose-load.

Though clinical trials are not usually conducted on over the counter (OTC) supplements, Charles Liu is committed to providing patients and healthcare professionals with assurance that the disclosed dietary supplement (which is expected to be marketed under the name “Diabetian C”) is a safe and effective supplement for diabetic and pre-diabetic patients. Based on the results of two studies to date, DiabetainC is effective at decreasing Hb A1c, fasting blood glucose and cholesterol. Results were compiled from raw data in the last 3 years by Charles Liu, Clinical Pharmacist.

In another aspect of this disclosure, a dietary supplement for improving health and reducing risks associated with Type 2 diabetes includes sources of magnesium and zinc. In general, a metal or mineral source in an amount equivalent to a reference compound refers to a molar equivalent of the mineral or metal, and includes the reference compound.

Magnesium ions are essential to the basic nucleic acid chemistry of life and are essential to all cells in all known living organisms. Individuals that are at risk of being diagnosed with Type 2 diabetes or that have been diagnosed with Type 2 diabetes typically have a magnesium deficiency. Accordingly, a source of biologically available magnesium is desirable. The magnesium source can be any magnesium compound that can be safely administered orally to provide magnesium ions for proper cell function. Examples include magnesium chloride, magnesium oxide, magnesium gluconate, magnesium malate, magnesium orotate, magnesium glycinate and magnesium citrate. A single dose for twice daily administration may contain the equivalent of from 10 mg to 400 mg of magnesium chloride (i.e., an amount of a magnesium source that provides magnesium that is equivalent to magnesium chloride, or the molar equivalent). For example, a single dose for twice daily administration can contain the equivalent of 10 mg, 20 mg, 50 mg, 100 mg, 200 mg, 300 mg or 400 mg of magnesium chloride.

Zinc possesses antioxidant properties, helps speed up the healing process and is beneficial to strengthening and protecting the immune system of humans and other mammals. Diabetes patients often have a zinc deficiency. As such, a source of biologically available zinc is also considered essential. The zinc source can be any zinc compound that can be safely administered orally to provide systemic absorption. Examples include zinc chloride, zinc oxide, zinc sulfate, zinc picolinate, zinc gluconate, zinc citrate and zinc glycinate. A single dose for twice daily administration may contain the equivalent of from 3 mg to 20 mg of zinc chloride (i.e., an amount of zinc compound that provide systemic absorption of zinc that is equivalent to zinc chloride). For example, a single dose for twice daily administration can contain the equivalent of 3 mg, 5 mg, 10 mg, 15 mg or 20 mg of zinc chloride.

In certain aspects of this disclosure, a source of chromium is included, along with the magnesium and zinc, in the dietary supplement. There is evidence that chromium is needed or desired to produce glucose tolerance factor, which was found to exert a beneficial insulin-mimetic and insulin-potentiating effect that is likely to be useful for correcting imbalances in glucose metabolism and treatment of diabetes patients. Thus, certain dietary supplements in accordance with this disclosure may contain a chromium compound that can be safely administered orally, twice daily, to provide systemic absorption of chromium in an amount equivalent to from about 50 micrograms (mcg) to about 500 mcg of chromium picolinate, e.g., 50 mcg, 75 mcg, 100 mcg, 200 mcg, 300 mcg, 400 mcg or 5 mcg. Examples include, in addition to chromium picolinate itself, chromium polynicotinate, chromium citrate, chromium chloride and chromium nicotinate.

In certain aspects of this disclosure, a dietary supplement includes Vitamin B12, in addition to a source of magnesium, zinc and optionally chromium. Vitamin B12 is important for maintaining normal function of the brain and nervous system, and for the formation of blood. It is normally involved in the metabolism of every cell of the human body, especially affecting DNA synthesis and regulation, and also involved in fatty acid metabolism and amino acid metabolism. It is believed that administration of Metformin reduces serum Vitamin B12 levels and long-term use of Metformin substantially increases the risk of Vitamin B12 deficiency and hyperhomocysteinemia, which is an independent risk factor for cardiovascular disease, especially among individuals with Type 2 diabetes. Therefore, certain dietary supplements in accordance with this disclosure can contain Vitamin B12 in an amount that is effective to correct a Metformin induced deficiency. A suitable amount in a single dosage form for twice daily administration is from about 2 mcg to about 100 mcg, e.g., 2 mcg, 3 mcg, 4 mcg, 5 mcg, 10 mcg, 20 mcg, 25 mcg, 50 mcg or 100 mcg.

In accordance with certain aspects of this disclosure, the dietary supplement may include one or more herbal powders or plant extracts, in addition to a source of magnesium and zinc, and in addition to optional Vitamin B12 and an optional source of chromium.

An extract or powder from American Ginseng may be added to the dietary supplements disclosed herein to help lower blood glucose levels in people with Type 2 diabetes. It is believed that American Ginseng slows the absorption of sugars from the diet. There is also evidence that American Ginseng may make cells more receptive to insulin in people who are insulin resistant. American Ginseng is also believed to reduce stress, strengthen the immune system, and decrease blood pressure. The amount of American Ginseng extract or powder can be sufficient to provide from about 10 mg to about 100 mg of ginsenosides in a single dosage form intended for twice daily administration. Suitable powdered American Ginseng and extracts (water, alcohol, or water and alcohol) are commercially available or can be prepared using conventional methods for preparing herbal powders and extracts. Ethanol and water extracts (e.g., 50-70% ethanol and 30% to 50% water) can be blended with the other components of the dietary supplement and optional excipients, such as binders, and compressed into tablet dosage forms or filled into hard or soft shell capsules.

An oil extract or powder of cinnamon bark may be added to the disclosed dietary supplements to work alone or in concert with insulin and/or Metformin to reduce blood glucose levels, thus reducing the need for insulin and/or better controlling blood glucose levels. A suitable amount of cinnamon bark, oil or extract that may be added to twice daily dosage forms is an amount that provides from about 500 mg to about 2500 mg of methylhydroxychalcone polymer, which is believed to be an insulin mimetic. For example, methylhydroxychalcone polymer can be provided in a twice daily dosage form in amounts of 500 mg, 1000 mg, 1500 mg, 2000 mg or 2500 mg.

Lutein can be added to the disclosed dietary supplements to protect against diabetic complications. Diabetes is often associated with complications, such as cataracts and increased susceptibility to frequent and protected infections. High glucose levels associated with diabetes induce oxidative stress in immune system cells and increase Nuclear Factor-kappa B activity linked to cancer, inflammatory diseases, autoimmune diseases, septic shock, and viral infection. A suitable amount of Lutein that can be added to a dietary supplement dosage form for twice daily administration is from about 1 mg to about 25 mg, e.g., 5, 10 or 20 mg.

Green tea extract can be added to the disclosed dietary supplements in an amount sufficient to provide a single dose from twice daily administration from about 100 mg to about 2000 mg of epigallocatechin gallate to reduce insulin resistance and/or to prevent, delay or retard the development of Type 2 diabetes. For example, epigallocatechin gallate can be provided in a twice daily dosage form in amounts of 100 mg, 200 mg, 300 mg, 400 mg, 500 mg, 1000 mg, 1500 mg and 2000 mg.

The sources of magnesium and zinc, along with an optional source of chromium III, Vitamin B12, American Ginseng, cinnamon bark, Lutein and green tea can be combined with fillers and/or excipients, such as flavorants, colorants, opacifiers, binders, disintegrants, lubricants, etc., and pressed into tablets or caplets, or filled into hard or soft shell capsules (e.g., gelatin capsules). While the disclosed dosage forms have been described as being for twice daily administration, the dosage forms can be formulated for more than twice daily administration, or for less than twice daily administration (e.g., daily administration), with amounts of the component ingredients being adjusted appropriately (not necessarily proportionally).

The dietary supplements disclosed herein are not intended as general purpose nutritional supplements, but are instead targeted to the treatment, prevention and/or amelioration of Type 2 diabetes. As such, the disclosed supplements may consist of, or consist essentially of a source of magnesium and a source of zinc, and optionally consist of or consist essentially of a source of chromium, Vitamin B12, American Ginseng, cinnamon bark, Lutein, green tea extract, and a pharmaceutically active agent for treatment of Type 2 diabetes (e.g., insulin). Therefore the tablets and capsules of this disclosure can be formulated so that they do not contain other vitamins or minerals commonly added to multiple vitamin and mineral tablets and capsules used for general purpose nutritional supplementation. This allows diabetes patients and those at risk for developing diabetes to take the disclosed supplements and optionally take other supplements, as needed or desired, generally without needing to consider excessive dosing of nutrients.

Although the natural extracts, powdered forms, and oils of plants previously described are believed to be highly beneficial and economical, it is possible that selected active agents could be isolated from the plant material or synthesized. Accordingly, in certain aspects of this disclosure, the dietary supplement may comprise, consist essentially of, or consist of a magnesium source, a zinc source, an optional chromium source, optional Vitamin B12, optional ginsenoside(s) (10 mg-100 mg for a twice daily tablet), optional methylhydroxychalcone polymer (500 mg-2500 mg for a twice daily tablet), optional lutein, and optional epigallocatechin gallate (100 mg-2000 mg for a twice daily tablet).

Clinical trials have shown that “DiabetainC” supplements in accordance with this disclosure, when administered to Type 2 diabetes patients, lowers Hb Ale by an average of 1.7% when co-administered with Metformin, as compared with a 1.2% lowering of Hb A1c when Metformin is used by itself. This is a significant improvement. A 0.5% to 1% lowering of Hb Al c correlates with a 20% reduction in morbidity. Clinical trials have also shown that on average co-administration of “DiabetainC” and other oral anti-diabetes medications reduces Hb A1c by 1.25% as compared with 0.75% when the other anti-diabetes medications are used alone.

Clinical trials have shown an advantageous decrease in fasting blood glucose (FBG) of 72 mg/dL when co-administered with Metformin, as compared with a lowering of 48 mg/dL when Metformin is administered alone. Clinical trials have also demonstrated a lowering of FBG by 45 mg/dL when co-administered with other anti-diabetes medications as compared with a lowering of FBG by 30 mg/dL when the other diabetes medications are administered alone. These improvements are significant, as every 15 mg/dL to 40 mg/dL decrease in FBG reduces diabetes related heart diseases up to 50%.

In another aspect of this disclosure, the tablets and capsules can further comprise an antidiabetes medication, such as Metformin, in well known effective amounts. For example, Metformin could be incorporated into a single dose for twice daily administration in an amount of 500 mg.

The dietary supplements disclosed herein for regulating glucose metabolism and treating or preventing Type 2 diabetes or pre-diabetes can be administered with or without insulin, with or without Metformin and/or other anti-diabetes medications, and can be potentiated or enhanced with prescribed dietary and/or lifestyle modifications. Such modifications that can reduce risks associated with Type 2 diabetes include reaching and maintaining a reasonable or normal body weight (e.g., a BMI below 25, or below 20), daily or regular physical activity (e.g., at least 20 or 30 minutes of exercise most days of the week), and limiting fat intake to about 25 percent of daily total calories.

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. A dietary supplement in capsule or tablet dosage form, comprising:
an orally administrable source of magnesium in an amount that is the equivalent of from 10 mg to 400 mg of magnesium chloride;
an orally administrable source of zinc in an amount that is the equivalent of from 3 mg to 20 mg of zinc chloride;
optionally an orally administrable source of chromium in an amount that is the equivalent of up to 500 micrograms of chromium picolinate;
optionally Vitamin B12 in an amount up to 100 micrograms;
optionally an extract or powdered form of American Ginseng in an amount that provides up to 100 mg of ginsenosides;
optionally an oil, extract or powdered form of cinnamon bark in an amount that provides up to 2500 mg of methylhydroxychalcone polymer;
optionally lutein in an amount up to 25 mg; and
optionally green tea extract in an amount that provides up to 2000 mg of epigallocatechin gallate.

2. The dietary supplement of claim 1, in which the orally administrable source of chromium is present in an amount that is the equivalent of at least 50 micrograms of chromium picolinate.

3. The dietary supplement of claim 2, in which Vitamin B12 is present in an amount of at least 2 micrograms.

4. The dietary supplement of claim 3, in which the extract or powdered form of American Ginseng is present in an amount that provides at least 10 mg of ginsenosides.

5. The dietary supplement of claim 4, in which the oil, extract or powdered form of cinnamon bark is present in an amount that provides at least 500 mg of methylhydroxychalcone polymer.

6. The dietary supplement of claim 5, in which lutein is present in an amount of at least 1 mg.

7. The dietary supplement of claim 6, in which the green tea extract is present in an amount that provides at least 100 mg of epigallocatechin gallate.

8. The dietary supplement of claim 1, in which the extract or powdered form of American Ginseng is present in an amount that provides at least 10 mg of ginsenosides.

9. The dietary supplement of claim 8, in which the oil, extract or powdered form of cinnamon bark is present in an amount that provides at least 500 mg of methylhydroxychalcone polymer.

10. The dietary supplement of claim 8, in which lutein is present in an amount of at least 1 mg.

11. The dietary supplement of claim 8, in which the green tea extract is present in an amount that provides at least 100 mg of epigallocatechin gallate.

12. The dietary supplement of claim 9, in which lutein is present in an amount of at least 1 mg.

13. The dietary supplement of claim 9, in which the green tea extract is present in an amount that provides at least 100 mg of epigallocatechin gallate.

14. The dietary supplement of claim 10, in which the green tea extract is present in an amount that provides at least 100 mg of epigallocatechin gallate.

15. The dietary supplement of claim 12, in which the green tea extract is present in an amount that provides at least 100 mg of epigallocatechin gallate.

16. A dietary supplement in capsule or tablet dosage form, consisting of:
an orally administrable source of magnesium in an amount that is the equivalent of from 10 mg to 400 mg of magnesium chloride;
an orally administrable source of zinc in an amount that is the equivalent of from 3 mg to 20 mg of zinc chloride;
optionally an orally administrable source of chromium in an amount that is the equivalent of up to 500 micrograms of chromium picolinate;
optionally Vitamin B12 in an amount up to 100 micrograms;
optionally up to 100 mg of at least one ginsenoside;
optionally up to 2500 mg of methylhydroxychalcone polymer;
optionally up to 25 mg of lutein;
optionally up to 2000 mg of epigallocatechin gallate; and
optionally excipients.

17. A method of treating, preventing or ameliorating Type 2 diabetes or treating pre-diabetes, comprising:
orally administering to a patient on a daily basis via one or a plurality of tablets or capsules at a single time or multiple times throughout the day, a source of magnesium in an amount equivalent to 20 mg to 800 mg of magnesium chloride; a source of zinc in an amount equivalent to 6 mg to 40 mg of zinc chloride; optionally a source of chromium in an amount equivalent to 100 micrograms to 1000 micrograms of chromium picolinate; optionally from 4 micrograms to 200 micrograms of Vitamin B12; optionally an extract or powdered form of American Ginseng in an amount that provides from 20 mg to 200 mg of ginsenoside(s), or 20 mg to 200 mg of isolated or synthesized ginsenoside(s); optionally an oil, extract or powdered form of cinnamon bark in an amount that provides 1000 mg to 5000 mg of methylhydroxychalcone polymer; optionally 2 mg to 50 mg lutein; and optionally green tea extract in an amount that provides from 200 mg to 4000 mg of epigallocatechin gallate or 200 mg to 4000 mg of isolated or synthesized epigallocatechin gallate; and
optionally prescribing dietary and lifestyle modifications that reduce risks associated with Type 2 diabetes.

18. The method of claim 17, further comprising administering to the patient an anti-diabetes medication.

19. The method of claim 17, further comprising administering to the patient insulin and an anti-diabetes medication.

20. The method of claim 17, further comprising administering to the patient Metformin.

21. The method of claim 17, further comprising administering to the patient insulin and Metformin.

22. The method of claim 17, further comprising administering to the patient insulin.

23. The method of claim 17, further comprising administering to the patient Metformin and another anti-diabetes medication.

24. A combination dietary supplement and medication in capsule or tablet form comprising:
an orally administrable source of magnesium in an amount that is the equivalent of from 10 mg to 400 mg of magnesium chloride;
an orally administrable source of zinc in an amount that is the equivalent of from 3 mg to 20 mg of zinc chloride;
optionally an orally administrable source of chromium in an amount that is the equivalent of up to 500 micrograms of chromium picolinate;
optionally Vitamin B12 in an amount up to 100 micrograms;
optionally an extract or powdered form of American Ginseng in an amount that provides up to 100 mg of ginsenosides;
optionally an oil, extract or powdered form of cinnamon bark in an amount that provides up to 2500 mg of methylhydroxychalcone polymer; and
an effective amount of an anti-diabetes medication.

25. The combination dietary supplement and medication of claim 24, in which the anti-diabetes medication is Metformin.

26. The combination dietary supplemental medication of claim 24, in which the anti-diabetes medication includes Metformin and at least one other anti-diabetes medication.
